(19) United States
(12) Patent Application Publication
(10) Pub. No.: US 2024/0206511 A1
(43) Pub. Date: Jun. 27, 2024

(54) DEVELOPMENT OF NATURAL SEASONING USING LACTIC ACID BACTERIA FERMENTED PRODUCT OF OYSTER MUSHROOM AND WINTER MUSHROOM

(71) Applicant: DAY.N BIO CORP., Jeollanam-do (KR)

(72) Inventors: KYOUNG-SUN SEO, Jeollanam-do (KR); KYUNG-JE KIM, Jeollanam-do (KR); SEONG-WOO JIN, Jeollanam-do (KR); YOUNG-WOO KOH, Jeollanam-do (KR); SEUNG-BIN IM, Jeollanam-do (KR); NEUL-I HA, Jeollanam-do (KR); HEE-GYEONG JEONG, Jeollanam-do (KR); SEUNG-EON BAN, Jeollanam-do (KR); YU-SEON KIM, Jeollanam-do (KR); SANG-WOOK JEONG, Jeollanam-do (KR); SEUNG-JU KIM, Jeollanam-do (KR); JEONG-HEE KIM, Jeollanam-do (KR); HEE-SOO KIM, Jeollanam-do (KR); BOK-SEON KIM, Jeollanam-do (KR); KI MAN KIM, Gwangju (KR); SE EUN PARK, Gwangju (KR); JUNHUI CHOI, Jeollanam-do (KR)

(21) Appl. No.: 18/288,098

(22) PCT Filed: Dec. 16, 2021
(86) PCT No.: PCT/KR2021/019199

§ 371 Date: Oct. 24, 2023

(30) Foreign Application Priority Data
Dec. 16, 2020 (KR) 10-2020-0176801

(51) Int. Cl. A23L 27/10; A23L 17/60; A23L 29/00; A23L 31/00; C12N 1/20; C12R 1/01; C12R 1/23

(52) U.S. Cl. CPC A23L 27/10; A23L 17/60; A23L 29/065; A23L 31/00; C12N 1/205; A23V 2400/113; A23V 2400/427; C12R 2001/01; C12R 2001/23

(57) ABSTRACT

In the method of producing natural seasoning according to an embodiment, hot water extraction of kelp is carried out, followed by drying to prepare kelp extract, hot water extraction of oyster mushroom is carried out to prepare oyster mushroom extract, hot water extraction of winter mushroom is carried out to prepare winter mushroom extract, the oyster mushroom extract is inoculated with Pediococcus pentosaceus strain, followed by fermentation to prepare lactic acid bacteria fermented product of oyster mushroom, the winter mushroom extract is inoculated with Lactobacillus acidophilus strain, followed by fermentation to prepare lactic acid bacteria fermented product of winter mushroom, and the lactic acid bacteria fermented product of oyster mushroom, the lactic acid bacteria fermented product of winter mushroom, and the kelp extract are mixed to produce natural seasoning.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/019199 filed on Dec. 16, 2021, which claims priority to the benefit of Korean Patent Application No. 10-2020-0176801 filed in the Korean Intellectual Property Office on Dec. 16, 2020, the entire contents of which are incorporated herein by reference.

### BACKGROUND

### 1. Technical Field

The present invention relates to a method of producing natural seasoning characterized in that natural seasoning is produced by including a step of mixing lactic acid bacteria fermented product of oyster mushroom, lactic acid bacteria fermented product of winter mushroom, and kelp extract, and also natural seasoning produced by the same method.

### 2. Background Art

Conventionally, seasonings are used as an additive that is added in small amount to give natural taste and flavor to food. In recent years, however, various products having enhanced taste and nutrition by containing higher amount of original ingredients are available as consumer products. For example, in South Korea, “Natural teabag spice containing non-processed ingredients” was commercially launched by Sajo Haepyo, and it is a product prepared by drying, as a whole, 100% non-processed Korean food ingredients and packaging the dried ingredients in an environmentally-friendly teabag.

Due to the great advantage like easiness and convenience of use in various foods without needing to add other condiments, spices, or the like, liquid seasoning is now widely used by consumers.

In South Korea, the seasoning market is currently in the fourth generation. During last several decades, seasonings of the first and second generations have been highly popular among Korean people. However, as sodium glutamate, which is called “MSG”, gradually gains a negative image of an unhealthy food, demand for sodium glutamate hit rock bottom.

Accordingly, natural seasoning was developed to compensate this problem, and it corresponds to the seasoning of the third generation. Natural seasoning is produced by adding powder of non-processed ingredients like beef and sardine, and thus it finally shed off the negative image of an unhealthy product. However, there is still a limitation that, as the taste of the seasoning is mixed with food itself, only insufficient umami taste is obtained.

Meanwhile, MEEWON as the first-generation seasoning is recently found at the top of internet search volume list in South Korea, since Koreans are wildly informed with the safety of MSG as it is proven by Ministry of Food and Drug Safety, and, as it is officially decided that the term “chemically synthesized additive” is removed from the classified category of food additives, a change in the consumer recognition of MSG is gradually caused. Furthermore, as homemade meal and cookbang (i.e., cooking broadcasting) become popular among young South Koreans, it is believed that the change is brought in response to the trend in which general seasoning, which is relatively cheaper than natural seasoning, is preferred when it comes to buying new seasonings.

Yeondoo (i.e., purely vegetable cooking essence prepared by fermenting beans and extracting vegetables) as the fourth-generation seasoning is also found at the top of internet search volume list in South Korea, and as there is a higher trend of pursuing good health and a higher number of so-called “checksumer”, who is sensitive about the components and non-processed ingredients or the like before busing any seasoning, seasonings having enhanced taste and flavor by containing natural ingredients are currently in high demand.

Mushroom is a healthy food containing various nutritional components like protein, lipid, sugar, mineral, amino acids, and vitamin. By having unique taste and flavor, it has been conventionally used for preparing meals. Furthermore, as it is known to have an anti-oxidation activity, an anti-bacterial activity, an anti-tumor activity, an anti-cancer activity, or the like, it currently draws high attention ever than before. In recent years, there is a need for developing new food materials that can contribute to the prevention of age-related disease like cancer, circulatory disease, and diabetes by using, in addition to the components which are considered to be useful from the viewpoint of food science, various functional components contained in mushroom. Accordingly, research and development regarding the selection and application method of food and microbial resources containing various functional components are needed more than ever.

From the year 1990, the efficacy of pharmaceutical mushrooms is widely known via various news media, and consumers quickly start to pay great attention on those mushrooms. However, on the negative side, there was much exaggerated advertising of the pharmaceutical mushrooms, and the consumer confidence decreases rapidly from late 2000. It has been already indicated by many studies that the pharmaceutical effect or functional property of edible mushrooms is 70% or so of the pharmaceutical mushrooms.

It is thus believed that, if various foods are prepared by utilizing many nutritional components and functional materials contained in edible mushrooms, they can be developed into a product which has competitiveness at home and abroad and also can contribute to the improvement of farm household income.

In Korea Patent Application Publication No. 2014-0002235, a method of producing natural mushroom seasoning is disclosed, and, in Korea Patent Registration No. 2119227, a method of producing fermented oyster mushroom seasoning is disclosed. However, those methods are different from the natural seasoning of the present invention using lactic acid bacteria fermented products of oyster mushroom and winter mushroom.

### SUMMARY

The present invention is devised under the circumstances that are described in the above, and object of the present invention is to provide a method of producing natural seasoning that can enhance the flavor and umami taste (i.e., sweet and savory taste) of food by selecting a bacterial strain suitable for fermentation of oyster mushroom and winter mushroom and mixing the lactic acid bacteria fermented products of oyster mushroom and winter mushroom with kelp extract in suitable amount.

To solve the problem described in the above, the present invention provides a method of producing natural seasoning characterized in that it is produced by including: (1) carrying out hot water extraction of kelp after adding water followed by drying to prepare kelp extract; (2) carrying out hot water extraction of oyster mushroom after adding water to prepare oyster mushroom extract; (3) carrying out hot water extraction of winter mushroom after adding water to prepare winter mushroom extract; (4) inoculating the oyster mushroom extract prepared in the step (2) with Pediococcus pentosaceus strain followed by fermentation to prepare lactic acid bacteria fermented product of oyster mushroom; (5) inoculating the winter mushroom extract prepared in the step (3) with Lactobacillus acidophilus strain followed by fermentation to prepare lactic acid bacteria fermented product of winter mushroom; and (6) mixing the lactic acid bacteria fermented product of oyster mushroom prepared in the step (4), the lactic acid bacteria fermented product of winter mushroom prepared in the step (5), and the kelp extract prepared in the step (1).

The present invention also provides natural seasoning produced by the aforementioned method.

The lactic acid bacteria fermented product of oyster mushroom and lactic acid bacteria fermented product of winter mushroom of the present invention, which are added to seasoning, can enhance the flavor and umami taste of food while maximizing the nutritional value of the mushrooms, and thus the seasoning prepared by mixing the fermented food materials with kelp extract in suitable amount can be advantageously used as a seasoning food that can increase the preference of food based on enhancement of the umami taste and flavor of food.

### BRIEF DESCRIPTION OF THE DRAWINGS

FIG. 1 is a graph for determining a change in pH of the fermented product of oyster mushroom depending on various bacterial strain types and different fermentation period.

FIG. 2 is a graph for determining a change in pH of the fermented product of winter mushroom depending on various bacterial strain types and different fermentation period.

FIG. 3 is a graph for determining a change in total acidity of the fermented product of oyster mushroom depending on various bacterial strain types and different fermentation period.

FIG. 4 is a graph for determining a change in total acidity of the fermented product of winter mushroom depending on various bacterial strain types and different fermentation period.

FIG. 5 is a graph for comparing DPPH radical scavenging activity of the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 6 is a graph for comparing hydroxyl radical scavenging activity of the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 7 is a graph for comparing the activity of inhibiting α-amylase by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 8 is a graph for comparing the activity of inhibiting β-glucosidase by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 9 is a graph for comparing the activity of inhibiting lipase by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 10 is a graph for comparing cytotoxicity of the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning) to Raw264.7 cells, in which all test samples have been tested at various concentrations.

FIG. 11 is a graph for comparing cytotoxicity of the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning) to RBL-2H3 cells, in which all test samples have been tested at various concentrations.

FIG. 12 is a graph for comparing cytotoxicity of the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning) to AGS cells, in which all test samples have been tested at various concentrations.

FIG. 13 is a graph for comparing the production amount of NO by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 14 is a graph for comparing the activity of inhibiting IL-1β by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 15 is a graph for comparing the activity of inhibiting TNF-α by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 16 is a graph for comparing the activity of inhibiting PGE2 by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 17 is a graph for comparing the 0-hexosaminidase activity of the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

FIG. 18 is a graph for comparing the activity of inhibiting histamine by the oyster mushroom extract (i.e., oyster mushroom), fermented product of oyster mushroom (i.e., oyster mushroom P), winter mushroom extract (i.e., winter mushroom), fermented product of winter mushroom (i.e., winter mushroom A), and natural seasoning (i.e., seasoning), in which all test samples have been tested at various concentrations.

### DETAILED DESCRIPTION

To achieve the purpose of the present invention, the present invention provides a method of producing natural seasoning characterized in that it is produced by including:
(1) carrying out hot water extraction of kelp after adding water followed by drying to prepare kelp extract;
(2) carrying out hot water extraction of oyster mushroom after adding water to prepare oyster mushroom extract;
(3) carrying out hot water extraction of winter mushroom after adding water to prepare winter mushroom extract;
(4) inoculating the oyster mushroom extract prepared in the step (2) with Pediococcus pentosaceus strain followed by fermentation to prepare lactic acid bacteria fermented product of oyster mushroom;
(5) inoculating the winter mushroom extract prepared in the step (3) with Lactobacillus acidophilus strain followed by fermentation to prepare lactic acid bacteria fermented product of winter mushroom; and
(6) mixing the lactic acid bacteria fermented product of oyster mushroom prepared in the step (4), the lactic acid bacteria fermented product of winter mushroom prepared in the step (5), and the kelp extract prepared in the step (1).

With regard to the method of producing natural seasoning of the present invention, Pediococcus pentosaceus strain mentioned above is a strain which has been deposited with Korean Collection for Type Cultures (KCPC) having the address of Korea Research Institute of Bioscience and Biotechnology (KRIBB), 181, Ipsin-gil Jeongeup-si, Jeollabuk-do, 56212, Republic of Korea) on Dec. 15, 2021 under the Accession number ofKCTC 14826BP. The deposit has been made under the terms of the Budapest Treaty and all restrictions imposed by the depositor on the availability to the public of the biological material will be irrevocably removed upon the granting of a patent.

Furthermore, Lactobacillus acidophilus strain mentioned above is a strain which has been deposited with Korean Collection for Type Cultures (KCPC) having the address of Korea Research Institute of Bioscience and Biotechnology (KRIBB), 181, Ipsin-gil Jeongeup-si, Jeollabuk-do, 56212, Republic of Korea) on Dec. 15, 2021 under the Accession number of KCTC 14825BP. The deposit has been made under the terms of the Budapest Treaty and all restrictions imposed by the depositor on the availability to the public of the biological material will be irrevocably removed upon the granting of a patent.

When the lactic acid bacteria fermented product of oyster mushroom and lactic acid bacteria fermented product of winter mushroom are produced by using the specific strains that have been deposited as described above, it is possible to prepare a fermented product which not only has excellent umami taste, high content of ergosterol and β-glucan, and increased amount of nucleic acid-related materials and amino acids such as aspartic acid and glutamic acid exhibiting umami taste but also shows, without exhibiting any cytotoxicity, enhanced anti-oxidation activity and high inhibitory effect on α-amylase and lipase, and an excellent anti-inflammatory activity by having enhanced activity of inhibiting NO, IL-1β, TNF-α, and prostaglandin E2 (PGE2), all causing an inflammation. Furthermore, by having an excellent effect of inhibiting β-hexosaminidase and histamine, a fermented product having enhanced anti-allergic activity can be prepared.

Furthermore, with regard to the method of producing natural seasoning described in the present invention, the kelp extract of the step (1) may be preferably prepared by adding 18 to 22 times (v/w) of water to kelp followed by hot water extraction for 2 to 4 hours at 75 to 85° C. and drying. More preferably, it may be prepared by adding 20 times (v/w) of water to kelp followed by hot water extraction for 3 hours at 80° C. and drying.

Furthermore, with regard to the method of producing natural seasoning described in the present invention, the oyster mushroom extract of the step (2) may be preferably prepared by adding 8 to 12 times (v/w) of water to oyster mushroom followed by hot water extraction for 3 to 5 hours at 75 to 85° C. More preferably, it may be prepared by adding 10 times (v/w) of water to oyster mushroom followed by hot water extraction for 4 hours at 80° C.

Furthermore, with regard to the method of producing natural seasoning described in the present invention, the winter mushroom extract of the step (3) may be preferably prepared by adding 8 to 12 times (v/w) of water to winter mushroom followed by hot water extraction for 3 to 5 hours at 85 to 95° C. More preferably, it may be prepared by adding 10 times (v/w) of water to winter mushroom followed by hot water extraction for 4 hours at 90° C.

When the extractions of kelp, oyster mushroom, and winter mushroom are carried out at the conditions of the steps (1), (2), and (3) above, more efficient extraction can be achieved due to higher solid content.

Furthermore, with regard to the method of producing natural seasoning described in the present invention, the fermentation of the steps (4) and (5) above may be preferably carried out for 2 to 4 days at 34 to 40° C., and it may be more preferably carried out for 3 days at 37° C. When the fermentation is carried out at the conditions described above, fermentation with enhanced flavor and umami taste can be obtained while unpleasant odor specific to mushroom is removed.

Still furthermore, with regard to the method of producing natural seasoning of the present invention, the mixing of the step (6) may be mixing, based on the total weight of natural seasoning, 45 to 55% by weight of the lactic acid bacteria fermented product of oyster mushroom, 27 to 33% by weight of the lactic acid bacteria fermented product of winter mushroom, and 18 to 22% by weight of the kelp extract. More preferably, it may be mixing, based on the total weight of natural seasoning, 50% by weight of the lactic acid bacteria fermented product of oyster mushroom, 30% by weight of the lactic acid bacteria fermented product of winter mushroom, and 20% by weight of the kelp extract. When the mixing is carried out at the aforementioned condition, harmonious existence of taste and flavor of various ingredients is obtained so that the ingredients can be prepared as seasoning that can enhance the umami taste of food.

More specifically, the method of producing natural seasoning of the present invention may include:
(1) carrying out hot water extraction of kelp for 2 to 4 hours at 75 to 85° C. after adding 18 to 22 times (v/w) of water followed by drying to prepare kelp extract;
(2) carrying out hot water extraction of oyster mushroom for 3 to 5 hours at 75 to 85° C. after adding 8 to 12 times (v/w) of water to prepare oyster mushroom extract;
(3) carrying out hot water extraction of winter mushroom for 3 to 5 hours at 85 to 95° C. after adding 8 to 12 times (v/w) of water to prepare winter mushroom extract;
(4) inoculating the oyster mushroom extract prepared in the step (2) with Pediococcus pentosaceus JMIL002 strain (KCTC 14826BP) followed by fermentation for 2 to 4 days at 34 to 40° C. to prepare lactic acid bacteria fermented product of oyster mushroom;
(5) inoculating the winter mushroom extract prepared in the step (3) with Lactobacillus acidophilus JMIL001 strain (KCTC 14825BP) followed by fermentation for 2 to 4 days at 34 to 40° C. to prepare lactic acid bacteria fermented product of winter mushroom; and
(6) mixing, based on the total weight of natural seasoning, 45 to 55% by weight of the lactic acid bacteria fermented product of oyster mushroom prepared in the step (4), 27 to 33% by weight of the lactic acid bacteria fermented product of winter mushroom prepared in the step (5), and 18 to 22% by weight of the kelp extract prepared in the step (1).

Even more specifically, the method of producing natural seasoning of the present invention may include:
(1) carrying out hot water extraction of kelp for 3 hours at 80° C. after adding 10 times (v/w) of water followed by drying to prepare kelp extract;
(2) carrying out hot water extraction of oyster mushroom for 4 hours at 80° C. after adding 10 times (v/w) of water to prepare oyster mushroom extract;
(3) carrying out hot water extraction of winter mushroom for 4 hours at 90° C. after adding 10 times (v/w) of water to prepare winter mushroom extract;
(4) inoculating the oyster mushroom extract prepared in the step (2) with Pediococcus pentosaceus JMIL002 strain (KCTC 14826BP) followed by fermentation for 3 days at 37° C. to prepare lactic acid bacteria fermented product of oyster mushroom;
(5) inoculating the winter mushroom extract prepared in the step (3) with Lactobacillus acidophilus JMIL001 strain (KCTC 14825BP) followed by fermentation for 3 days at 37° C. to prepare lactic acid bacteria fermented product of winter mushroom; and
(6) mixing, based on the total weight of natural seasoning, 50% by weight of the lactic acid bacteria fermented product of oyster mushroom prepared in the step (4), 30% by weight of the lactic acid bacteria fermented product of winter mushroom prepared in the step (5), and 20% by weight of the kelp extract prepared in the step (1).

The present invention further provides natural seasoning produced by the aforementioned method.

Hereinbelow, the present invention is explained in greater detail in view of the Examples. However, the following Preparation examples and Examples are given only for illustration of the present invention and it is evident that the scope of the present invention is not limited by those Preparation examples and Examples.

### Preparation Example 1. Natural Seasoning

(1) By carrying out hot water extraction for 3 hours at 80° C. after adding 20 times (v/w) of water to dried kelp followed by drying, kelp extract was prepared.
(2) By carrying out hot water extraction for 4 hours at 80° C. after adding 10 times (v/w) of water to oyster mushroom, oyster mushroom extract was prepared.
(3) By carrying out hot water extraction for 4 hours at 90° C. after adding 10 times (v/w) of water to winter mushroom, winter mushroom extract was prepared.
(4) After adding sugar and water to the oyster mushroom extract prepared in the step (2) for adjustment to 8 brix, inoculation with 10% Pediococcus pentosaceus JMIL002 strain (KCTC 14826BP) was carried out followed by fermentation for 72 hours at 37° C. to prepare lactic acid bacteria fermented product of oyster mushroom.
(5) After adding sugar and water to the winter mushroom extract prepared in the step (3) for adjustment to 8 brix, inoculation with 1% Lactobacillus acidophilus JMIL001 strain (KCTC 14825BP) was carried out followed by fermentation for 72 hours at 37° C. to prepare lactic acid bacteria fermented product of winter mushroom
(6) Based on the total weight of natural seasoning, 50% by weight of the lactic acid bacteria fermented product of oyster mushroom prepared in the step (4), 30% by weight of the lactic acid bacteria fermented product of winter mushroom prepared in the step (5), and 20% by weight of the kelp extract prepared in the step (1) were admixed with one another.

### 1. Experimental Method

### (1) Analysis of General Components

General components were analyzed according to the AOAC method, and the test items include moisture content, crude ash content, crude proteins, crude lipids, crude fibers, and soluble nitrogen-free materials. Specifically, moisture content was obtained by direct drying at 105° C. and crude ash content was obtained by direct ashing at 550° C. Furthermore, the crude proteins were calculated by multiplying the nitrogen amount, which has been measured by micro-Kjeldahl method, by 6.25, crude lipids were obtained by Soxhlet's extraction method, and content of crude fibers was obtained according to revised Henneberg Stohamann method. Content of the soluble nitrogen-free materials in sample was obtained by subtracting the contents of crude ash, crude proteins, crude lipids, and crude fibers from the total weight.

### (2) Analysis of Free Sugar

Distilled water was added to 5 g of sample. After homogenizing them using a homogenizer followed by stirring and leaching, the resultant was adjusted to 100 mL and centrifuged (3,000 rpm, 30 minutes). Then, purification using Sep-pak Cis was carried out and the filtered solution obtained after filtering through a 0.45 μm membrane filter (Millipore Co., USA) was analyzed by HPLC (High Performance Liquid Chromatography). The analysis conditions were as described in the following Table 1 and the content was calculated based on the external standard method.

TABLE 1
HPLC conditions for analysis of free sugar

| | |
|---|---|
| Item | Conditions for analysis |
| Device | Agilent Technologies 1200 Series |
| | ELSD detector |
| Column | ZORBAX Carbohydrate |
| | (4.6 × 150 mm) |
| Solvent | 85% Acetonitrile |
| Column temperature | 30° C. |
| Flow rate | 1.4 mL/min |
| Injection volume | 20 μL |

### (3) Analysis of Ergosterol

For analyzing ergosterol, 5 g of sample were added with 100 mL ethanol followed by reflux extraction for 1 hour at 80° C. Then, the supernatant was collected and the residuals were added with 100 mL ethanol followed by reflux extraction for 1 hour at 80° C. The extract was filtered, added with 20 mL ethanol and 10 g of potassium hydroxide followed by reflux extraction for 1 hour at 80° C. Then, the resulting gummed solution was added with 50 mL of distilled water. After that, fractionation with hexane was carried out 3 times, 50 mL for each, and the hexane layer was collected and fully concentrated. The resultant was then dissolved in 2 mL of methanol and examined by HPLC. Content was calculated based on the external standard method, and the HPLC conditions are the same as those described in the following Table 2.

TABLE 2
HPLC conditions for ergosterol analysis

| | |
|---|---|
| Item | Conditions for analysis |
| Device | Agilent Technologies 1200 Series |
| Column | Agilent XDB-C₁₈ (Method Development Kit) |
| | (4.6 × 150 mm, 5 μm) |
| Solvent | 98% Methanol |
| Column temperature | 28.8° C. |
| Wavelength | UV 280 nm |
| Flow rate | 1.0 mL/min |
| Injection amount | 20 μL |

### (4) Analysis of β-Glucan

Beta glucan in sample was measured by using Megazyme kit (Mushroom and Yeast β-glucan Assay Procedure K-YBGL, Megazyme, Ireland).

First, to measure the total glucan, 100 mg of the crushed sample obtained after sifting through a 100 mesh sieve were added to a tube, and then, after adding 1.5 mL of 37% HCl to the sample, degraded for 45 minutes in a water bath at constant-temperature of 30° C. After that, distilled water was added in an amount of 10 mL followed by vortexing and culture for 2 hours at 100° C. While cooling to room temperature, the mixture was added with 10 mL of 2 N KOH and adjusted to 100 mL with 200 mM sodium acetate buffer followed by thorough mixing. Thereafter, to 0.1 mL of the supernatant, 0.1 mL of exo-1,3-β-glucanase plus 0-glucosidase dissolved in 200 mM sodium acetate buffer was added while 0.2 mL of acetate buffer was added for the reagent blank. As D-glucose standard, 0.1 mL of D-glucose standard and 0.1 mL of acetate buffer were mixed with each other, and culture was carried out for 60 minutes at 40° C. Next, GOPOD (glucose oxidase/peroxidase mixture) was added in an amount of 3 mL and culture was carried out for 20 minutes at 40° C. Absorbance was measured at 510 nm.

To measure α-glucan, 100 mg of the crushed sample obtained after sifting through a 100 mesh sieve were added to a tube, and then, after adding 2 mL of 2 M KOH, the mixture was mixed for 20 minutes. After adding 8 mL of 1.2 M sodium acetate buffer followed by addition of 0.2 mL of amyloglucosidase plus invertase and thorough mixing, culture was carried out for 30 minutes in a water bath at constant-temperature of 40° C. Thereafter, to 0.1 mL of the supernatant, 0.1 mL of 200 mM sodium acetate buffer and 3 mL of GOPOD were added, and culture was continued for 20 minutes at 40° C. Absorbance was then measured at 510 nm. By subtracting the content of α-glucan from the content of total glucan, content of β-glucan was quantified.

### (5) Analysis of Nucleic Acid-Related Materials

About 2 g of sample was weighed and added with 10 mL of 10%-HClO₄. After homogenization and filling in a test tube to 25 mL, the mixture was allowed to stand for 30 minutes and then filtered. pH of the supernatant was adjusted, and the pH-adjusted solution was brought to 50 mL by using 10%-HClO₄. After allowing the mixture to stand for 30 minutes, it was filtered through a 0.4 μm filter and then subjected to HPLC analysis. As a column, U-Bondapak C18 (4 mm×30 cm) was used. Elution was made with detector wavelength of 265 nm and 30% methanol as a solvent. With regard to the quantification, GMP (5′-guanosine cyclic monophosphate), IMP (5′-inosine mono phosphate), and XMP (5′-xanthylic acids) were quantified based on the external standard method.

### (6) Constitutional Amino Acids

Sample (0.5 g) was added to a test tube and, after adding 10 mL of 6 N—HCl solution and sealing of the tube tip by melting with hot flame to give an ampoule, subjected to hydrolysis for 24 hours in an autoclave at 110° C. The ampoule was then broken, filtered using a filtered paper, and adjusted to 50 mL with methanol. After concentration under reduced pressure, it was adjusted to 5 mL of 20 mM HCl followed by filtration using a 0.45 μm membrane filter. The obtained filtered solution was collected in a small amount and then derivatized using the AccQ-Tag reagents. Thereafter, it was analyzed by HPLC.

### (7) Free Amino Acids

For the analysis of free amino acids in a sample, 10 mL of the solution, which has been obtained by the quantification of free sugar above, was added with 25 mg of sulfosalicylic acid and the mixture was allowed to stand for 4 hours at 4° C. Proteins were then removed by centrifuge (50000 rpm, 30 minutes) and the filtered solution, which has been obtained by filtering the supernatant through a 0.45 μm membrane filter, was then derivatized then using the AccQ-Tag reagents. Thereafter, it was analyzed by HPLC. Conditions for the analysis are as described in the following Table 3.

TABLE 3
Conditions for amino acid analysis

| Item | Conditions for analysis | | | |
|---|---|---|---|---|
| Device | Agilent Technologies 1200 Series | | | |
| Detector | Agilent Technologies 1200 Series FLD | | | |
| Column | AccQ-Tag ™ | | | |
| | (Waters Co., 150 mm L. × 3.9 mm I.D.) | | | |
| Column | 37° C. | | | |
| temperature | | | | |
| Buffer solution | A: AccQ-Tag Eluent A (Acetate-Phosphate buffer) | | | |
| | B: AccQ-Tag Eluent B (100% acetonitrile) | | | |
| | C: D.W | | | |
| | Time | A | B | C |
| | 0 | 100 | 0 | 0 |
| | 0.5 | 99 | 1 | 0 |
| | 18 | 95 | 5 | 0 |
| | 19 | 91 | 9 | 0 |
| | 26 | 86.7 | 13.3 | 0 |
| | 30 | 84 | 16 | 0 |
| | 32 | 83 | 17 | 0 |
| | 36 | 0 | 60 | 40 |
| | 39 | 100 | 0 | 0 |
| | 48 | 100 | 0 | 0 |

| | |
|---|---|
| Flow rate | 1.0 mL/min |
| Injection amount | 5 μL |

### (8) Sensory Evaluation

Sensory test was carried out with 30 people who satisfied the panel requirement described in Korean Food Code. Requirement regarding the age and sex was fully satisfied. With regard to the items for preference evaluation, 7-point scoring method was employed for color, flavor, taste, and overall preference. Criteria for evaluation were as follows: very good—7; moderate—4; very bad—1. Number of the test sample was changed with an interval of 2 hours, and the test was repeated 3 times with the same panel. For each repeated test, the highest and lowest scores were excluded to calculate the average score.

### (9) Activity of α-Amylase

Sample at various concentrations was mixed with α-amylase enzyme solution originating from saliva (5 unit/mL, in 50 mM potassium phosphate buffer) followed by reaction for 5 minutes at room temperature. Then, 1% starch as a substrate solution was added to the mixture. After stirring, the reaction was allowed to occur for 5 minutes at room temperature. After the reaction, the mixture was added with 3,5-DNS (3,5-dinitrosalicylic acid) and boiled for 5 minutes at 100° C. to have a chromogenic reaction. Then, after cooling and stirring with addition of DW, the absorbance at 550 nm was measured by using a microplate reader (Molecular Devices, Sunnyvale, CA, USA) to evaluate the activity of α-amylase.

### (10) Activity of α-Glucosidase

Rat intestinal acetone powder (30 mg) was mixed with 1 mL of 50 mM potassium phosphate buffer, which has been previously reacted at 4° C., and dissolved in the buffer. After centrifuge for 10 minutes at 4° C. and 4,000 rpm, the middle layer was used as α-glucosidase enzyme solution. Sample at various concentrations was mixed with α-glucosidase enzyme solution (50 μL) and 3 mM ρ-NPG (ρ-nitrophenyl-α-D-glucopyranoside, 100 μL). After the reaction for 60 minutes at 37° C., absorbance at 405 nm was measured by using a microplate reader (Molecular Devices, Sunnyvale, CA, USA) for evaluation.

### (11) Lipolytic Activity Assay

By using p-NPB (p-nitrophenyl butyrate) as a substrate (10 mM), a spectrophotometric assay was carried out. After reacting the sample prepared at various concentrations with a substrate solution for 15 minutes at 60° C., PNP (p-nitrophenol) was measured for 10 minutes at 405 nm, and analyzed Vmax value. Measurement was carried out by using a microplate reader (Molecular Devices, Sunnyvale, CA, USA).

### (12) Determination of Stomach-Protecting Effect

### A) Cell Culture

For RAW264.7 cells, RBL-2H3 cells, and AGS cells, DMEM medium containing 10% FBS and 1× antibiotic-antimycotic agent was used. All cells were cultured at conditions of 37° C., 5% CO₂. When the cells were grown to 70% to 80% level of the culture flask, they were subjected to subculture using trypsin-EDTA.

### B) Measurement of Cytotoxicity

Cytotoxicity was measured by using MTT (3-(4,5-dimethylthiazole-2-yl)-2,5-diphenyl-tetrazoliumbromide) reduction analysis. Specifically, cells were aliquoted in a 96-well plate and cultured for 12 hours. Then, the cells were treated with each test sample and cultured again for 24 hours. After that, MTT solution (final concentration: 0.5 mg/mL) was added and the cells were further cultured for 4 hours at 37° C. to reduce MTT. Then, the medium was removed such that formazan generated by reduction of MTT is not released into the medium. After aliquoting DMSO in an amount of 10 μL and followed by mixing for 10 minutes, absorbance at 540 nm was measured.

### (13) Determination of Anti-Inflammatory and Anti-Allergic Effect

### A) Measurement of Production Amount of NO (Nitric Oxide)

Cells were aliquoted onto a 24-well plate and cultured for 12 hours. Then, the cells were treated with each test sample at various concentrations and, after a treatment with LPS (1 μg/mL), cultured again for 20 hours. Cell culture broth (100 μL) and Griess reagent (100 μL) were admixed with each other, and then the cells were reacted with the mixture for 10 minutes at room temperature. Then, absorbance at 550 nm was measured by using an ELISA reader. After establishing a standard curve using sodium nitrate, NO content was calculated.

### B) Inhibitory Activity on β-Hexosaminidase Secretion

By using DMEM medium containing DNP-IgE (dinitrophenyl-immunoglobulin E) with concentration of 0.5 μg/mL, cells were aliquoted onto a 24-well plate such that they are present at density of 2×10⁵ cells/mL in each well and cultured for 12 hours in 5% CO₂ incubator at 37° C. Each cell was then washed twice with Siraganian buffer (119 mM NaCl, 5 mM KCl, 5.6 mM glucose, 0.4 mM MgCl2, 25 mM PIPES, 1 mM CaCl₂), 0.1% BSA, pH 7.2), and then pre-cultured for 10 minutes using the same buffer in 5% CO₂ incubator at 37° C. After that, each test sample was diluted to various concentrations and reacted with the cells for 30 minutes in 5% CO₂ incubator at 37° C. Then, DNP-BSA (DNP-bovine serum albumin) was added such that it has final concentration of 100 ng/ml, and the cells were reacted for 2 hours in 5% CO₂ incubator at 37° C. After further culture for 10 minutes in an ice bath, the reaction was terminated. Supernatant (40 μL) was transferred to a 96-well plate, added with 40 μL of substrate buffer (2 mM 4-p-nitrophenyl-N-acetyl-β-D-glucosaminide, 0.05 M Sodium citrate, pH 4.5) followed by reaction for 1 hour at 37° C. By adding the termination solution (0.1 M Na₂CO₃/NaHCO₃, pH 10.5) in an amount of 200 μL per each well, the reaction was terminated and absorbance at 405 nm was measured.

### C) Inhibitory Activity on Histamine Secretion

Cultured cells were centrifuged for 5 minutes at 4° C., 2000 rpm, and the supernatant was used as a test sample. To a 1.5 mL tube, 25 μL of test sample was added, which was further added with 0.1 N HCl (22.5 μL) and 60% HClO₄ (42.5 μL) followed by mixing and centrifuge. Thus-obtained supernatant (40 μL) was added to a tube in which 5 N NaOH (25 μL), n-butanol (500 μL), and NaCl (0.06 g) are admixed with one another. After shaking and centrifuge (2000 rpm, 10 min), the butanol layer (400 μL) was transferred to a fresh 1.5 mL tube and 0.1 N HCl solution (150 μL) and n-heptane (0.5 mL) were added thereto followed by shaking and centrifuge. To thus-obtained aqueous layer (100 μL), 1 N NaOH solution (200 μL) and 0.1% o-phthaldialdehyde solution (5 μL) were added. After mixing and reacting them for 3 minutes at 37° C., 3 N HCl solution (10 μL) was further added and mixed. The mixture was allowed to stand for 2 minutes, and absorbance at 360 nm (excitation) and 450 nm (emission) was measured.

### D) Measurement of Prostaglandin E2

AGS cells were adjusted to have a concentration of 1×10⁴ cells/well, and, after the pre-culture for 1 hour with a test sample, the test was carried out. Indomethacin was used as a control. Quantification was carried out by using PGE2 assay kit.

### E) Measurement of Cytokine

To a 96-well plate which has been previously coated, primary antibody of cytokine to be quantified was added in an amount of 100 μL, and it was allowed to stand overnight at 4° C. On the next day, it was washed 3 times, each for 5 minutes, with 0.5% Tween 20 washing solution, and, after adding the sample for measurement and standard solution, each in an amount of 100 μL, the reaction was allowed to occur for 2 hours at room temperature. After the washing, HRP-conjugated secondary antibody was added in an amount of 100 μL and reacted for 2 hours at room temperature. After washing again the mixture, avidin/biotin were added to have chromogenic reaction, and absorbance was measured. From the absorbance value of standards, a standard curve was established, which was then used for quantifying the amount of cytokine based on the absorbance of each sample.

### (14) Statistical Processing

With the obtained test results, mean value and standard deviation among different test groups were calculated by using SPSS statistical analysis program (SPSS, ver. 16.0, USA). The statistical significance was at the significance level of p<0.05, and the obtained results were examined by Duncan's multiple range test in one-way ANOVA.

### Example 1. Kelp Yield Depending on Various Extraction Conditions

Kelp yield depending on various kelp extraction solvents and extraction temperatures are described in the following Table 4. As a result, it was found that the highest solid content is obtained from the 80° C. hot water extract. Accordingly, in the present study, the 80° C. hot water extract was used as a test sample after drying.

TABLE 4
Kelp yield depending on various conditions for extracting dried kelp (%)

| | Solid content (g) | | | | | |
|---|---|---|---|---|---|---|
| | | 60° C. Hot | 70° C. Hot | 80° C. Hot | 90° C. Hot | |
| | Water | water | water | water | water | Ethanol |
| Dried kelp 100 g | 8.5 | 14.5 | 26.8 | 34.1 | 28.4 | 18.2 |

### Example 2. Oyster Mushroom Yield and Winter Mushroom Yield Depending on Various Extraction Conditions

Oyster mushroom yield and winter mushroom yield depending on various extraction conditions are described in the following Table 5. For the oyster mushroom, it was found that the highest solid content is obtained from the 80° C. hot water extract. For the winter mushroom, it was found that the highest solid content is obtained from the 90° C. hot water extract. Accordingly, in the present study, the oyster mushroom extract obtained by 80° C. hot water extraction and winter mushroom extract obtained by 90° C. hot water extraction were used as a plant-based lactic acid bacteria fermented material.

TABLE 5
Oyster mushroom yield and winter mushroom yield
depending on various extraction solvents (%)

| | Solid content (g) | | | | | |
|---|---|---|---|---|---|---|
| | | 60° C. Hot | 70° C. Hot | 80° C. Hot | 90° C. Hot | |
| Category | Water | water | water | water | water | Ethanol |
| Oyster mushroom 100 g | 0.4 | 2.7 | 5.2 | 10.1 | 8.4 | 3.2 |
| Winter mushroom 100 g | 0.6 | 1.9 | 4.8 | 8.7 | 9.9 | 2.7 |

### Example 3. Analysis of Plant-Based Lactic Acid Bacteria Fermented Product of Oyster Mushroom and Winter Mushroom

### 1) Sensory Test

Result of the sensory test which has been carried out with regard to the umami taste of lactic acid bacteria fermented product of oyster mushroom and winter mushroom by following 5-point scoring method was shown in Table 6 given below. Specifically, it was found that more enhanced umami taste is obtained from the lactic acid bacteria fermented product of oyster mushroom and winter mushroom compared to the oyster mushroom extract and winter mushroom extract. With regard to oyster mushroom, high preference regarding umami taste was obtained from the fermented product resulting from fermentation with Pediococcus pentosaceus JMIL002 strain, and, with regard to winter mushroom, the high preference was obtained from the fermented product resulting from fermentation with Lactobacillus acidophilus JMIL001 strain.

TABLE 6
Evaluation of umami taste of oyster mushroom extract
and fermented product of various strain types

| | Oyster | Winter |
|---|---|---|
| Strain type | mushroom | mushroom |
| Extract | 2.80 | 3.02 |
| Lactobacillus plantarum | 3.68 | 3.16 |
| Leuconostoc lactis | 3.34 | 3.90 |
| Lactobacillus acidophilus JMIL001 | 3.50 | 4.50 |
| Lactobacillus acidophilus 2 | 3.78 | 3.52 |
| Lactobacillus sakei LI011 | 3.00 | 3.64 |
| Lactobacillus sakei LI033 | 3.42 | 3.80 |
| Lactobacillus sakei LGy039 | 3.00 | 3.82 |
| Pediococcus pentosaceus ALI015 | 3.04 | 4.08 |
| Pediococcus pentosaceus ALI024 | 3.82 | 3.78 |
| Pediococcus pentosaceus JMIL002 | 4.44 | 4.00 |

### 2) Measurement of pH

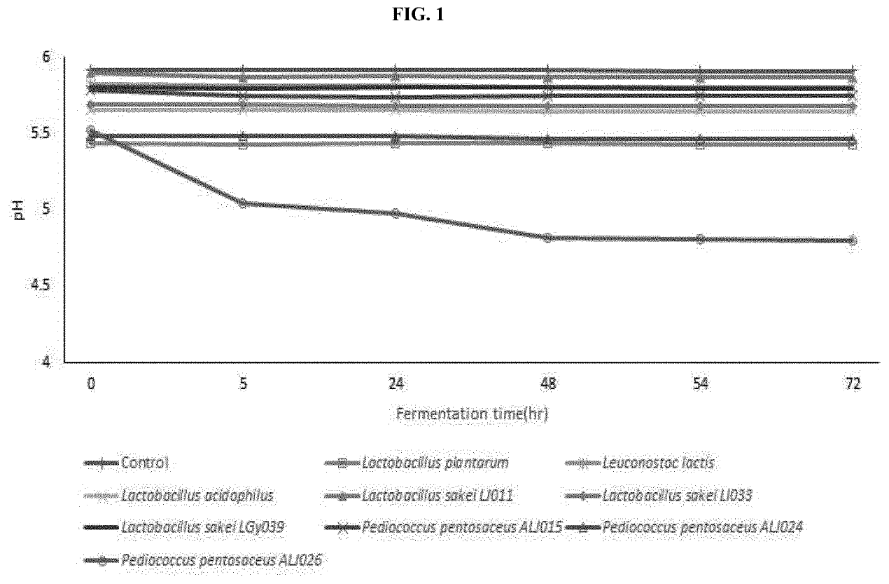

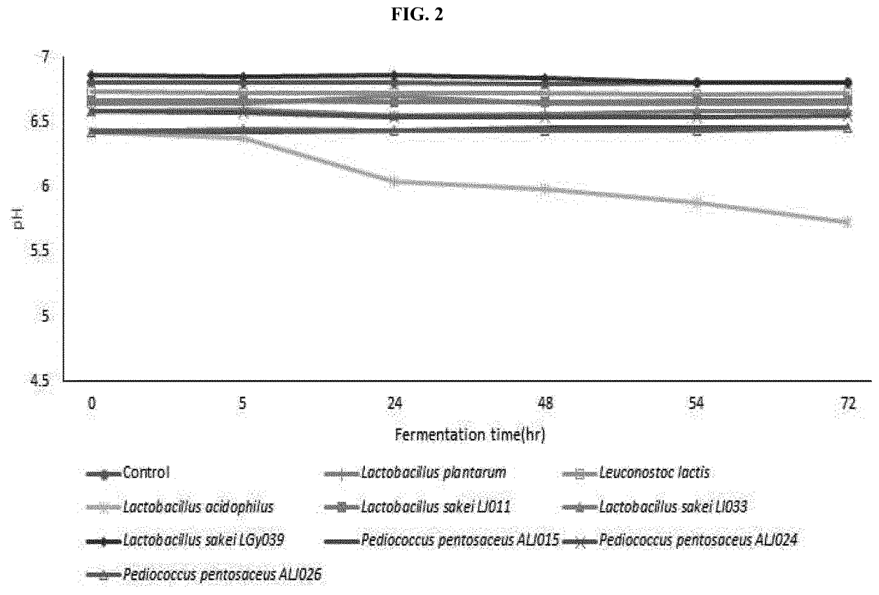

Oyster mushroom extract and winter mushroom extract were inoculated with various types of bacterial strains, and then pH was measured. As a result, it was found that remarkably lower pH is obtained from the lactic acid bacteria fermented product of oyster mushroom extract which has been inoculated with Pediococcus pentosaceus JMIL002 strain, i.e., from pH 5.525 to pH 4.794 (FIG. 1). In case of the lactic acid bacteria fermented product of winter mushroom extract, it was found that remarkably lower pH is obtained from the lactic acid bacteria fermented product of winter mushroom extract which has been inoculated with Lactobacillus acidophilus JMIL001 strain, i.e., from pH 6.425 to pH 5.732 (FIG. 2).

### 3) Measurement of Total Acidity

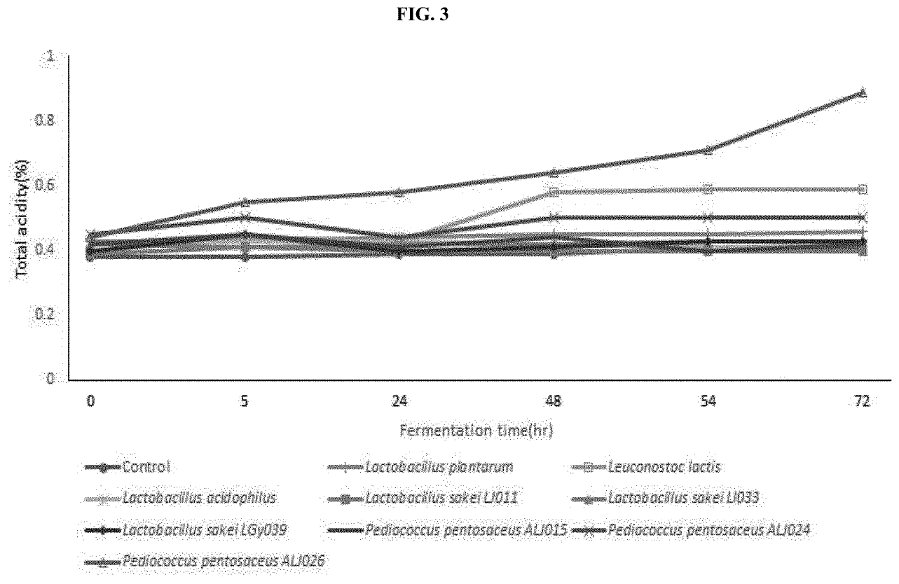

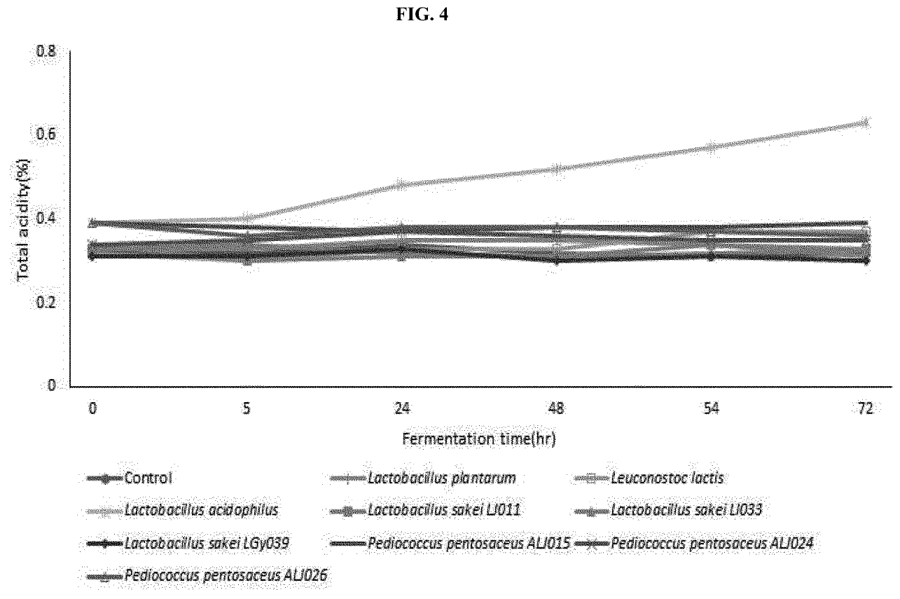

Oyster mushroom extract and winter mushroom extract were inoculated with various types of bacterial strains, and then total acidity was measured. As a result, it was found that remarkably higher total acidity is obtained from the lactic acid bacteria fermented product of oyster mushroom extract which has been inoculated with Pediococcus pentosaceus JMIL002 strain, i.e., pH of from 0.44 to 0.89 (FIG. 3). In case of the lactic acid bacteria fermented product of winter mushroom extract, it was found that remarkably higher total acidity is obtained from the lactic acid bacteria fermented product of winter mushroom extract which has been inoculated with Lactobacillus acidophilus JMIL001 strain, i.e., pH of from 0.39 to 0.63 (FIG. 4).

Accordingly, in the present study, Pediococcus pentosaceus JMIL002 strain and Lactobacillus acidophilus JMIL001 strain, which have been found to be suitable for the fermentation of oyster mushroom and winter mushroom for having excellent taste as well as smooth lactic acid bacteria fermentation, were selected as a final bacterial strain for fermentation.

### Example 4. Sensory Test Depending on Mixing Ratio of Natural Seasoning

By using the natural seasoning prepared by varying the mixing ratio of materials of the step (6) in Preparation example 1, a sensory test was carried out based on 7-point scoring method for 30 people who are qualified for a sensory test.

TABLE 7
Mixing ratio of natural seasoning

| | Addition ratio (% by weight) | | |
|---|---|---|---|
| | Lactic acid bacteria | Lactic acid bacteria | |
| | fermented product of | fermented product of | Kelp |
| Example | oyster mushroom | winter mushroom | extract |
| A | 70 | 10 | 20 |
| B | 60 | 20 | 20 |
| C | 50 | 30 | 20 |
| D | 40 | 40 | 20 |
| E | 30 | 50 | 20 |

As a result, it was found that the highest preference is obtained when the mixing is carried out with 50% by weight of the lactic acid bacteria fermented product of oyster mushroom, 30% by weight lactic acid bacteria fermented product of winter mushroom, and 20% by weight of kelp extract.

TABLE 8
Sensory test depending on mixing ratio of natural seasoning

| Type of natural | | | Overall |
|---|---|---|---|
| seasoning | Flavor | Taste | preference |
| A | 5.20 | 5.34 | 4.46 |
| B | 5.72 | 5.90 | 5.85 |
| C | 6.60 | 6.82 | 6.70 |
| D | 5.80 | 5.20 | 5.10 |
| E | 4.86 | 4.14 | 4.16 |

### Example 5. Content of General Components

Result of analyzing the content of general components in natural seasoning of Preparation example 1, oyster mushroom extract, lactic acid bacteria fermented product of oyster mushroom, winter mushroom extract, and lactic acid bacteria fermented product of winter mushroom is described in the following Table 9. As a control of the lactic acid bacteria fermented product, extract of each mushroom was used. As a result, it was found that there is no significant difference in terms of the content of moisture, ashes, crude lipids, and crude fibers. However, in terms of the crude proteins, a significant increase was shown from the oyster mushroom, i.e., 17.34% from oyster mushroom and 14.48% from winter mushroom. When compared to Control, all of the lactic acid bacteria fermented product showed a decrease in moisture, ashes, and crude fibers but with a slight increase in ashes, crude proteins, and crude lipids.

TABLE 9
Result of analysis of general components (%)

| | | | Crude | Crude | Crude | Soluble nitrogen-free |
|---|---|---|---|---|---|---|
| Category | Moisture | Ashes | proteins | lipids | fibers | extract¹⁾ |
| Natural seasoning | 8.62 | 3.45 | 21.48 | 1.56 | 7.76 | 57.13 |
| Oyster mushroom | 8.32 | 2.93 | 17.34 | 1.3 | 8.84 | 61.27 |
| extract | | | | | | |
| Fermented product of | 7.83 | 2.81 | 19.76 | 1.57 | 8.18 | 59.85 |
| oyster mushroom | | | | | | |
| Winter mushroom | 7.07 | 3.45 | 14.48 | 1.38 | 7.54 | 66.08 |
| extract | | | | | | |
| Fermented product of | 6.74 | 3.33 | 16.94 | 1.55 | 7.36 | 64.08 |
| winter mushroom | | | | | | |

¹⁾100 - (Total content of moisture, crude proteins, crude lipids, and crude ashes)

### Example 5. Content of Free Sugar

Result of analyzing the content of free sugar in natural seasoning of Preparation example 1, oyster mushroom extract, lactic acid bacteria fermented product of oyster mushroom, winter mushroom extract, and lactic acid bacteria fermented product of winter mushroom is described in the following Table 10. As a control of the lactic acid bacteria fermented product, extract of each mushroom was used. It was found that the oyster mushroom and winter mushroom contain only glucose and fructose, while the lactic acid bacteria fermented product additionally contains sucrose. Compared to Control, there was a significant decrease in glucose and fructose in the fermented product while sucrose was newly detected. In this regard, it is believed that reduced sugar was utilized as a carbon source by the microorganism during the fermentation.

TABLE 10
Analysis result of free sugar (%)

| Category | Glucose | Fructose | Sucrose | Total |
|---|---|---|---|---|
| Natural seasoning | 4.85 ± 0.48 | 1.54 ± 0.22 | 3.55 ± 0.39 | 6.39 ± 0.75 |
| Oyster mushroom extract | 2.54 ± 0.27 | 0.27 ± 0.05 | — | 2.81 ± 0.18 |
| Fermented product of | 1.05 ± 0.36 | 0.11 ± 0.09 | 1.48 ± 0.25 | 2.64 ± 0.33 |
| oyster mushroom | | | | |
| Winter mushroom extract | 1.73 ± 0.31 | 0.21 ± 0.04 | — | 1.94 ± 0.49 |
| Fermented product of | 0.64 ± 0.24 | 0.08 ± 0.05 | 1.27 ± 0.44 | 1.99 ± 0.53 |
| winter mushroom | | | | |

### Example 6. Content of Ergosterol

Result of analyzing ergosterol in natural seasoning of Preparation example 1, oyster mushroom extract, lactic acid bacteria fermented product of oyster mushroom, winter mushroom extract, and lactic acid bacteria fermented product of winter mushroom is described in the following Table 11. As a control of the lactic acid bacteria fermented product, extract of each mushroom was used. Ergosterol content was 284.19 mg % in the oyster mushroom and 232.45 mg % in the winter mushroom, showing a significantly higher value in the oyster mushroom. Compared to Control, the lactic acid bacteria fermented product showed a significant increase in the ergosterol content. As a precursor of vitamin D2, ergosterol converts to vitamin D2 upon irradiation of ultraviolet ray. It is known that the amount of ergosterol varies depending on types of mushroom. It is expected that the lactic acid bacteria fermented product, which contains a high amount of ergosterol known to have a physiological activity like conversion to vitamin D2, exhibits an effect of boosting immunity.

TABLE 11
Analysis result of ergosterol (mg %)

| Category | Ergosterol |
|---|---|
| Natural seasoning | 297.06 ± 49.2 |
| Oyster mushroom extract | 284.19 ± 19.96 |
| Fermented product of oyster mushroom | 334.85 ± 36.85 |
| Winter mushroom extract | 232.45 ± 34.27 |
| Fermented product of winter mushroom | 267.19 ± 54.6 |

### Example 7. Content of β-Glucan

Result of analyzing β-glucan in natural seasoning of Preparation example 1, oyster mushroom extract, lactic acid bacteria fermented product of oyster mushroom, winter mushroom extract, and lactic acid bacteria fermented product of winter mushroom is described in the following Table 12. As a control of the lactic acid bacteria fermented product, extract of each mushroom was used. R-Glucan content was 34.51% in the oyster mushroom and 19.37% in the winter mushroom, showing a significantly higher value in the oyster mushroom. Compared to Control, the lactic acid bacteria fermented product showed a significant increase. As one of the major physiologically active materials and one type of polysaccharides contained in mushroom, β-glucan is reported to exhibit an effect on human immune system and play a role of lowering blood sugar and controlling blood pressure.

TABLE 12
Analysis result of β-glucan (%)

| Category | Content |
|---|---|
| Natural seasoning | 28.74 ± 1.43 |
| Oyster mushroom extract | 34.51 ± 0.76 |
| Fermented product of oyster mushroom | 35.94 ± 1.51 |
| Winter mushroom extract | 19.37 ± 0.68 |
| Fermented product of winter mushroom | 21.26 ± 1.09 |

### Example 8. Content of Nucleic Acid Materials

Result of analyzing nucleic acid-related materials in natural seasoning of Preparation example 1, oyster mushroom extract, lactic acid bacteria fermented product of oyster mushroom, winter mushroom extract, and lactic acid bacteria fermented product of winter mushroom is described in the following Table 13. As a control of the lactic acid bacteria fermented product, extract of each mushroom was used. From both the oyster mushroom and winter mushroom, 5′-GMP, 5′-IMP, and 5′-XMP were detected as a nucleic acid material. Overall, the oyster mushroom appeared to contain a larger amount of nucleic acid materials. 5′-GMP content was the highest in both mushrooms, i.e., 108.93 mg % in the oyster mushroom and 95.26 mg % in the winter mushroom. Compared to Control, the lactic acid bacteria fermented product showed a slight increase in the nucleic acid-related materials. Regarding the nucleic acid-related materials, it is known that 5′-GMP exhibits the strongest taste while 5′-XMP is almost tasteless. It is also reported that content of nucleic acid-related materials varies depending on drying method and drying temperature.

TABLE 13
Analysis result of nucleic acid-related materials (mg %)

| | | | | Total nucleic acid |
|---|---|---|---|---|
| Category | 5′-GMP¹⁾ | 5′-IMP²⁾ | 5′-XMP³⁾ | related compounds |
| Natural seasoning | 132.56 ± 13.15 | 38.63 ± 1.7 | 69.48 ± 6.44 | 240.67 ± 21.09 |
| Oyster mushroom | 108.93 ± 26.96 | 33.48 ± 5.71 | 66.48 ± 11.24 | 208.89 ± 34.58 |
| extract | | | | |
| Fermented product of | 118.38 ± 19.38 | 40.61 ± 5.38 | 70.28 ± 8.62 | 229.27 ± 17.63 |
| oyster mushroom | | | | |
| Winter mushroom | 95.26 ± 11.29 | 31.47 ± 4.32 | 63.86 ± 8.41 | 190.59 ± 22.93 |
| extract | | | | |
| Fermented product of | 104.19 ± 14.45 | 38.26 ± 3.81 | 68.7 ± 12.36 | 211.15 ± 30.58 |
| winter mushroom | | | | |

¹⁾5′-Guanosine cyclic monophosphate
²⁾5′-inosine mono phosphate
³⁾5′-Xanthylic acids

### Example 9. Content of Constitutional Amino Acids

Result of analyzing constitutional amino acids in natural seasoning of Preparation example 1, oyster mushroom extract, lactic acid bacteria fermented product of oyster mushroom, winter mushroom extract, and lactic acid bacteria fermented product of winter mushroom is described in the following Table 14. As a control of the lactic acid bacteria fermented product, extract of each mushroom was used. Amino acids are a crucial nutrient constituting human body and, to maintain a healthy state of human body, it is required to obtain the amino acids from outside. Among the total 16 types of amino acids, arginine, glutamic acid, and valine appeared to be a major amino acid. Total content of the constitutional amino acids was 16830.58 mg % in the oyster mushroom extract and 10825.19 mg % in the winter mushroom extract, showing a significantly higher value in the oyster mushroom. Specifically, total content of the 8 types of amino acids, i.e., threonine, valine, methionine, lysine, isoleucine, leucine, histidine, and phenylalanine as an essential amino acid, was 7112.05 mg % in the oyster mushroom extract and 5207.09 mg % in the winter mushroom extract. It was recognized that, when the lactic acid bacteria fermented product is compared to the extract, a significant is obtained from both the oyster mushroom and winter mushroom.

TABLE 14
Analysis result of constitutional amino acids (mg %)

| | | Oyster | Fermented | Winter | Fermented |
|---|---|---|---|---|---|
| Total amino | Natural | mushroom | product of | mushroom | product of |
| acids | seasoning | extract | oyster mushroom | extract | winter mushroom |
| Aspartic acid | 454.05 ± 16.51 | 526.84 ± 50.23 | 635.74 ± 19.65 | 321.17 ± 27.24 | 382.69 ± 36.85 |
| Serine | 984.64 ± 28.96 | 1081.62 ± 38.25 | 1165.97 ± 85.22 | 841.65 ± 63.64 | 904.37 ± 27.23 |
| Glutamic acid | 1753.93 ± 57.18 | 2154.47 ± 83.48 | 2421.3 ± 74.96 | 1233.39 ± 67.43 | 1445.82 ± 89.31 |
| Glycine | 764.41 ± 11.28 | 1043.55 ± 42.08 | 1255.68 ± 14.28 | 445.26 ± 45.87 | 505.89 ± 62.14 |
| Histidine | 783.38 ± 43.83 | 881.26 ± 23.74 | 963.55 ± 41.94 | 625.5 ± 65.97 | 667.24 ± 57.49 |
| Arginine | 2920.53 ± 57.49 | 3764.13 ± 132.81 | 3852.26 ± 69.29 | 1956.93 ± 71.93 | 2208.51 ± 87.46 |
| Threonine | 912.78 ± 21.68 | 1049.26 ± 77.89 | 1144.16 ± 56.66 | 732.29 ± 77.9 | 798.21 ± 85.08 |
| Alanine | 137.76 ± 19.09 | 164.59 ± 22.98 | 188.94 ± 14.06 | 104.93 ± 18.86 | 122.36 ± 9.69 |
| Proline | 514.89 ± 15.61 | 582.93 ± 28.06 | 643.08 ± 31.38 | 426.84 ± 37.89 | 486.5 ± 10.32 |
| Tyrosine | 354.17 ± 27.17 | 400.4 ± 13.15 | 467.79 ± 28.75 | 287.93 ± 5.95 | 345.19 ± 27.94 |
| Valine | 1156.44 ± 38.22 | 1332.22 ± 82.36 | 1381.31 ± 56.11 | 920.66 ± 101.77 | 1057.53 ± 55.55 |
| Methionine | 230.02 ± 28.48 | 260.84 ± 33.21 | 305.58 ± 23.48 | 177.21 ± 10.74 | 211.43 ± 12.31 |
| Lysine | 460.43 ± 57.6 | 522.74 ± 15.94 | 597.23 ± 20.84 | 388.11 ± 13.8 | 439.85 ± 33.07 |
| Isoleucine | 567.54 ± 44.93 | 981.62 ± 65.09 | 1086.34 ± 28.2 | 717.46 ± 119.67 | 766.88 ± 13.84 |
| Leucine | 881.42 ± 55.74 | 955.45 ± 94.24 | 1063.91 ± 35.57 | 755.38 ± 55.65 | 834.07 ± 46.03 |
| Phenylalanine | 1019.57 ± 40.19 | 1128.66 ± 76.39 | 1235.4 ± 49.28 | 890.48 ± 71.62 | 961.28 ± 53.65 |
| TAA¹⁾ | 15361.46 ± 106.93 | 16830.58 ± 91.25 | 18408.24 ± 102.91 | 10825.19 ± 117.58 | 12137.82 ± 121.27 |
| EAA²⁾ | 6655.51 ± 81.35 | 7112.05 ± 56.16 | 7777.48 ± 76.54 | 5207.09 ± 113.54 | 5736.49 ± 68.88 |
| EAA/TAA | 43.33 ± 1.15 | 42.26 ± 2.15 | 42.25 ± 1.43 | 48.1 ± 1.28 | 47.26 ± 0.76 |
| (%)³⁾ | | | | | |

¹⁾TAA, total amino acid
²⁾EAA, essential amino acid (Thr + Val + Met + Ile + Leu + His + Lys)
³⁾EAA/TAA (%), essential amino acid/total amino acid

### Example 10. Content of Free Amino Acids

Result of analyzing free amino acids in natural seasoning of Preparation example 1, oyster mushroom extract, lactic acid bacteria fermented product of oyster mushroom, winter mushroom extract, and lactic acid bacteria fermented product of winter mushroom is described in the following Table 15. As a control of the lactic acid bacteria fermented product, extract of each mushroom was used. Among the total 16 types of amino acids, arginine, glutamic acid, and serine appeared to be a major amino acid. Total content of the free amino acids was 3910.32 mg % in the oyster mushroom and 2273.42 mg % in the winter mushroom, showing a significantly higher value in the oyster mushroom. Compared to Control, the lactic acid bacteria fermented product showed a significant increase. Specifically, total content of the 8 types of amino acids, i.e., threonine, valine, methionine, lysine, isoleucine, leucine, histidine, and phenylalanine as an essential amino acid, was 1994.98 mg % in the oyster mushroom and 1145.34 mg % in the winter mushroom. It was recognized that, when the lactic acid bacteria fermented product is compared to the content of free amino acids in Control, a significant increase is obtained from both the oyster mushroom and winter mushroom. Meanwhile, taste of food is generally decided by free amino acids. Aspartic acid and glutamic acid are known as a component exhibiting the umami taste of a fermented product while leucine and isoleucine may exhibit an influence on bitter taste. Based on the result of this study, it is believed that the flavor and taste of a lactic acid bacteria fermented product is greatly affected by the amino acids present in large amount.

TABLE 15
Analysis result of free amino acids (mg %)

| | | Oyster | Fermented | Winter | Fermented |
|---|---|---|---|---|---|
| Free amino | Natural | mushroom | product of | mushroom | product of |
| acids | seasoning | extract | oyster mushroom | extract | winter mushroom |
| Aspartic acid | 127.51 ± 29.75 | 112.47 ± 23.92 | 123.7 ± 21.19 | 64.44 ± 6.7 | 71.86 ± 7.35 |
| Serine | 498.87 ± 20.48 | 394.87 ± 20.13 | 462.22 ± 29.85 | 194.74 ± 17.33 | 282.24 ± 9.09 |
| Glutamic acid | 701.45 ± 38.66 | 604.93 ± 29 | 645.63 ± 39.16 | 413.86 ± 10.27 | 442.59 ± 2.77 |
| Glycine | 221.33 ± 13.73 | 193.41 ± 68.5 | 203.57 ± 23.17 | 107.32 ± 10.12 | 115.89 ± 10.18 |
| Histidine | 407.16 ± 26.28 | 383.19 ± 4.32 | 387.49 ± 2.67 | 218.8 ± 4.2 | 273.87 ± 3.58 |
| Arginine | 413.14 ± 13.29 | 360.29 ± 13.57 | 400.69 ± 7.11 | 159.03 ± 10.18 | 178.01 ± 9.33 |
| Threonine | 384.11 ± 15.24 | 322.14 ± 40.18 | 349.09 ± 0.13 | 221.21 ± 14.25 | 289.14 ± 2.14 |
| Alanine | 121.74 ± 11.57 | 91.26 ± 29.18 | 119.62 ± 4.02 | 64.44 ± 14.94 | 86.56 ± 13.18 |
| Proline | 94.93 ± 6.9 | 71.42 ± 6.48 | 84.7 ± 6 | 59.42 ± 8.56 | 62.66 ± 9.32 |
| Tyrosine | 102.11 ± 16.36 | 86.69 ± 20.77 | 92.19 ± 0.64 | 64.83 ± 10.27 | 70.82 ± 5.41 |
| Valine | 206.34 ± 35.54 | 183.27 ± 18.11 | 195.44 ± 11.99 | 130.79 ± 20.24 | 156.06 ± 14.75 |
| Methionine | 122.01 ± 4.79 | 103.03 ± 10.8 | 117.43 ± 21.03 | 56.41 ± 3.35 | 74.3 ± 12.72 |
| Lysine | 404.23 ± 21.15 | 349.09 ± 17.41 | 387.49 ± 29.27 | 126.66 ± 11 | 140.4 ± 18.72 |
| Isoleucine | 213.95 ± 26.59 | 172.28 ± 12.47 | 193.38 ± 13.7 | 93.65 ± 16.73 | 101.18 ± 8.81 |
| Leucine | 202.98 ± 11.57 | 187.38 ± 23.07 | 190.85 ± 7.97 | 99.79 ± 10.46 | 105.6 ± 10.45 |
| Phenylalanine | 313.58 ± 20.03 | 294.6 ± 27.82 | 306.91 ± 21.51 | 198.03 ± 18.67 | 237.96 ± 15.86 |
| TAA¹⁾ | 4535.44 ± 131.86 | 3910.32 ± 283.51 | 4260.4 ± 169.92 | 2273.42 ± 132.99 | 2689.14 ± 82.76 |
| EAA²⁾ | 2254.36 ± 124.66 | 1994.98 ± 109.57 | 2128.08 ± 78.53 | 1145.34 ± 143.57 | 1378.51 ± 80.79 |
| EAA/TAA | 49.71 ± 1.46 | 51.02 ± 2.14 | 49.95 ± 2.28 | 50.38 ± 1.98 | 51.26 ± 1.73 |
| (%)³⁾ | | | | | |

¹⁾TAA, total amino acid
²⁾EAA, essential amino acid (Thr + Val + Met + Ile + Leu + His + Lys)
³⁾EAA/TAA (%), essential amino acid/total amino acid

### Example 11. Analysis Result of Physiological Activity

### 1) Analysis Result of DPPH Radical Scavenging Activity

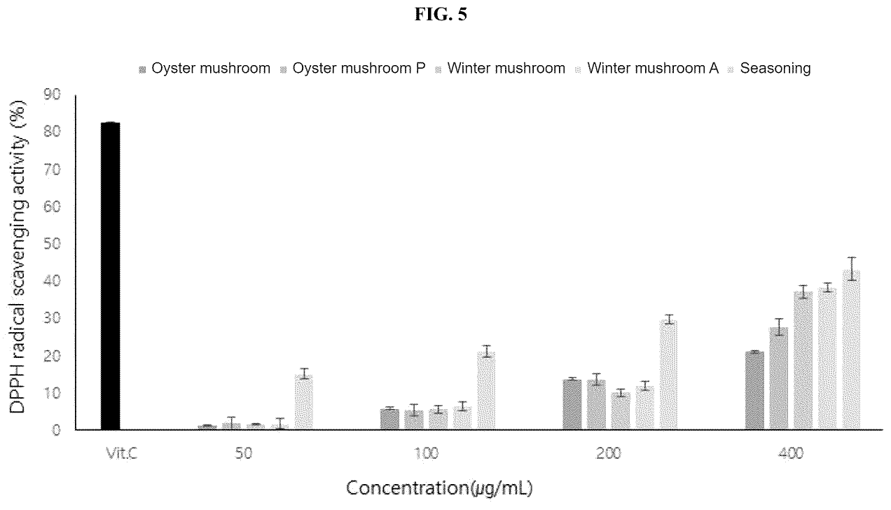

DPPH radical scavenging activity method is a method for evaluating the activity of a test sample for scavenging free radicals or donating hydrogens by a chemically-induced and relatively stable radical. Result of analyzing DPPH radical scavenging activity of oyster mushroom extract, fermented product of oyster mushroom, winter mushroom extract, fermented product of winter mushroom, and natural seasoning is illustrated in FIG. 5. At concentration of 400 μg/mL, the fermented product exhibited higher electron-donating ability compared to the non-processed ingredients. Specifically, the fermented product of oyster mushroom exhibited the electron donating ability of 27.7%, fermented product of winter mushroom exhibited the electron donating ability of 38.2%, and natural seasoning exhibited the electron donating ability of 43.3%.

### 2) Analysis Result of Hydroxyl Radical Scavenging Activity

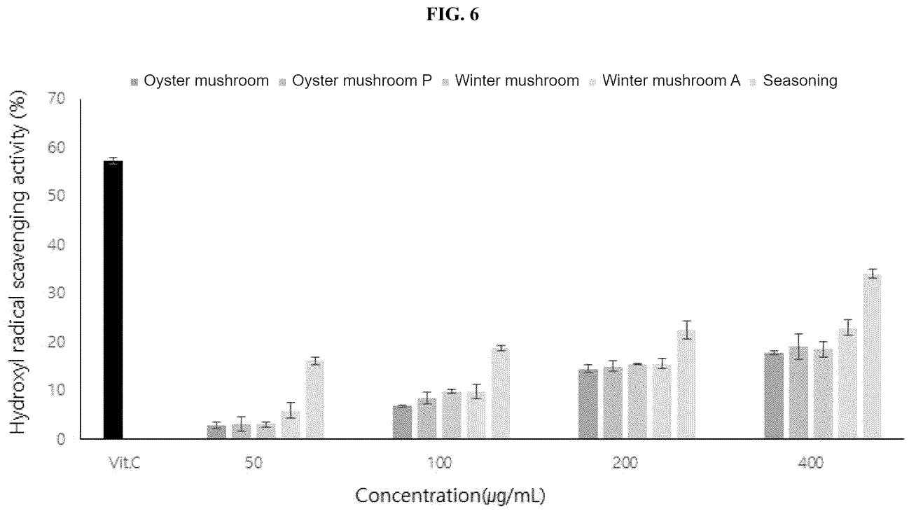

It is known that hydroxyl radical has the highest reactivity among active oxygen species and causes a great damage on neighboring biomolecules. Result of analyzing hydroxyl radical scavenging activity of oyster mushroom extract, fermented product of oyster mushroom, winter mushroom extract, fermented product of winter mushroom, and natural seasoning is illustrated in FIG. 6. As a result, it was found that the fermented product has higher scavenging activity compared to the samples of non-processed ingredient. Specifically, at concentration of 400 μg/mL, the oyster mushroom exhibited the scavenging activity of 17.8%, fermented product of oyster mushroom exhibited the scavenging activity of 19.1%, winter mushroom exhibited the scavenging activity of 18.5%, fermented product of winter mushroom exhibited the scavenging activity of 23.0%, and natural seasoning exhibited the scavenging activity of 34.1%.

### 3) Analysis Result of Inhibitory Activity on α-Amylase

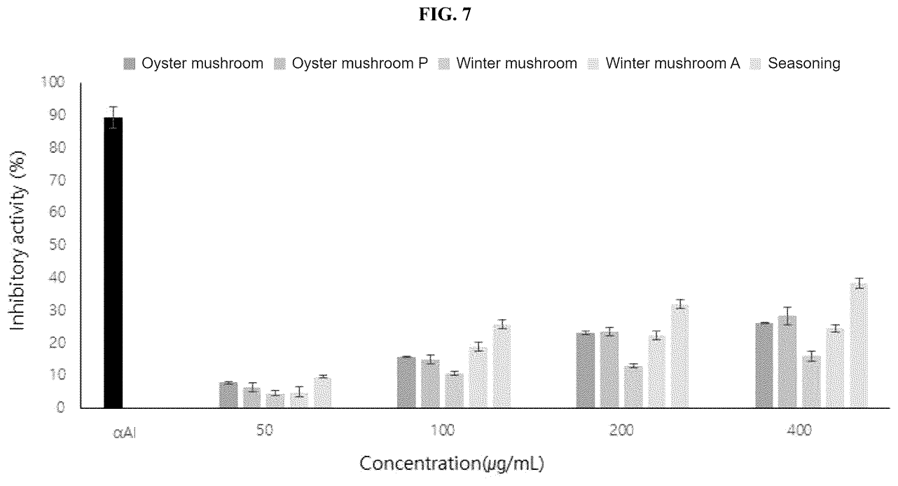

Result of analyzing inhibitory activity on α-amylase by oyster mushroom extract, fermented product of oyster mushroom, winter mushroom extract, fermented product of winter mushroom, and natural seasoning is illustrated in FIG. 7. At concentration of 400 μg/mL, the oyster mushroom exhibited the inhibitory activity of 26.2%, fermented product of oyster mushroom exhibited the inhibitory activity of 28.2%, winter mushroom exhibited the inhibitory activity of 15.8%, fermented product of winter mushroom exhibited the inhibitory activity of 24.4%, and natural seasoning exhibited the inhibitory activity of 38.3%.

### 4) Analysis Result of Inhibitory Activity on α-Glucosidase

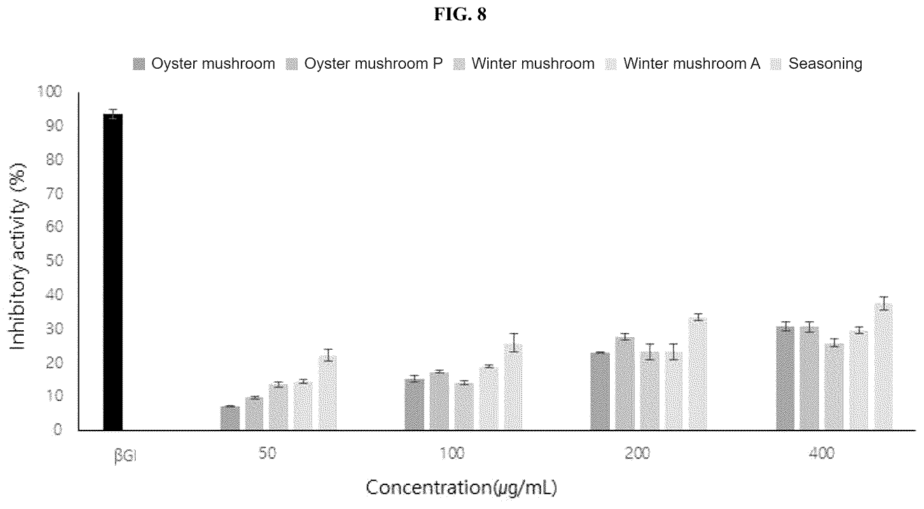

Result of analyzing inhibitory activity on α-glucosidase by oyster mushroom extract, fermented product of oyster mushroom, winter mushroom extract, fermented product of winter mushroom, and natural seasoning is illustrated in FIG. 8. At concentration of 400 μg/mL, the oyster mushroom exhibited the inhibitory activity of 30.6%, fermented product of oyster mushroom exhibited the inhibitory activity of 30.6%, winter mushroom exhibited the inhibitory activity of 25.8%, fermented product of winter mushroom exhibited the inhibitory activity of 29.4%, and natural seasoning exhibited the inhibitory activity of 37.6%.

### 5) Analysis Result of Inhibitory Activity on Lipase

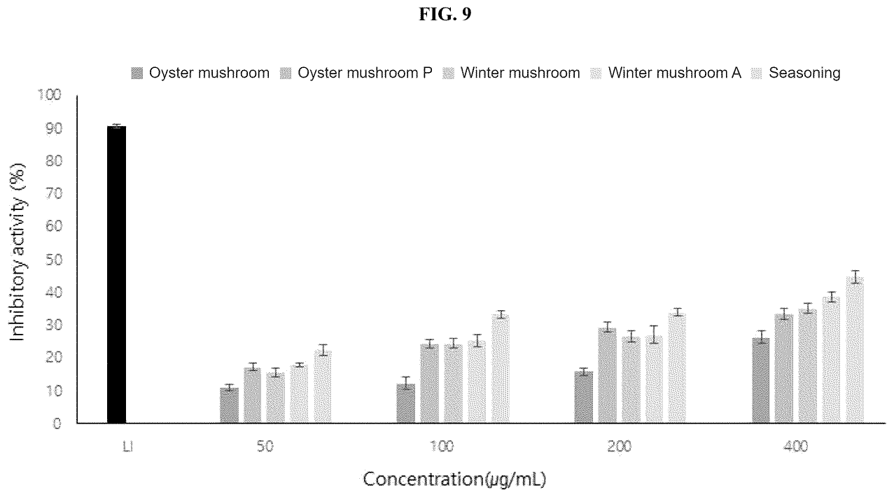

Result of analyzing inhibitory activity on lipase by oyster mushroom extract, fermented product of oyster mushroom, winter mushroom extract, fermented product of winter mushroom, and natural seasoning is illustrated in FIG. 9. At concentration of 400 μg/mL, the oyster mushroom exhibited the inhibitory activity of 26.4%, fermented product of oyster mushroom exhibited the inhibitory activity of 33.6%, winter mushroom exhibited the inhibitory activity of 35.2%, fermented product of winter mushroom exhibited the inhibitory activity of 38.7%, and natural seasoning exhibited the inhibitory activity of 44.8%.

### Example 12. Result of Cytotoxicity Analysis

### 1) Result of Cytotoxicity Analysis for Raw 264.7 Cells

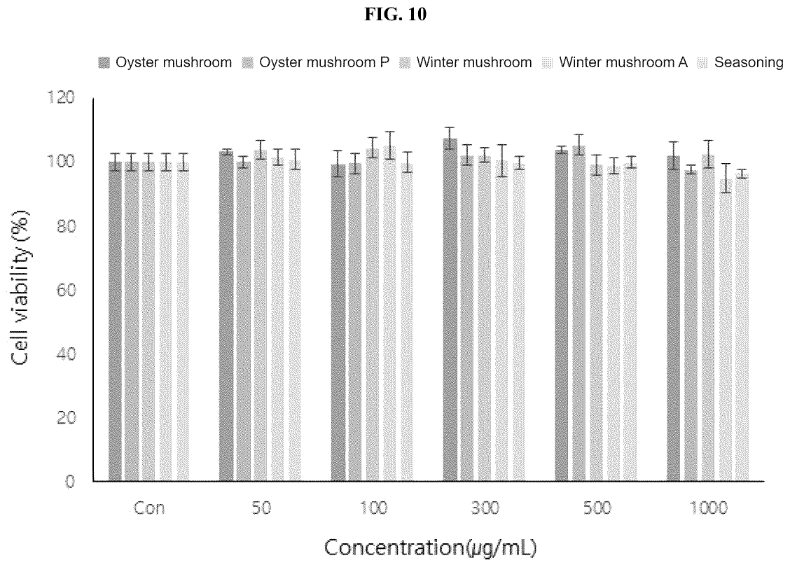

Result of analyzing cytotoxicity of the oyster mushroom extract, fermented product of oyster mushroom, winter mushroom extract, fermented product of winter mushroom, and natural seasoning on Raw 264.7 cells is the same as those illustrated in FIG. 10. Accordingly, it was found that no cytotoxicity on Raw 264.7 cells is exhibited by any test sample.

### 2) Result of Cytotoxicity Analysis for RBL-2H3 Cells

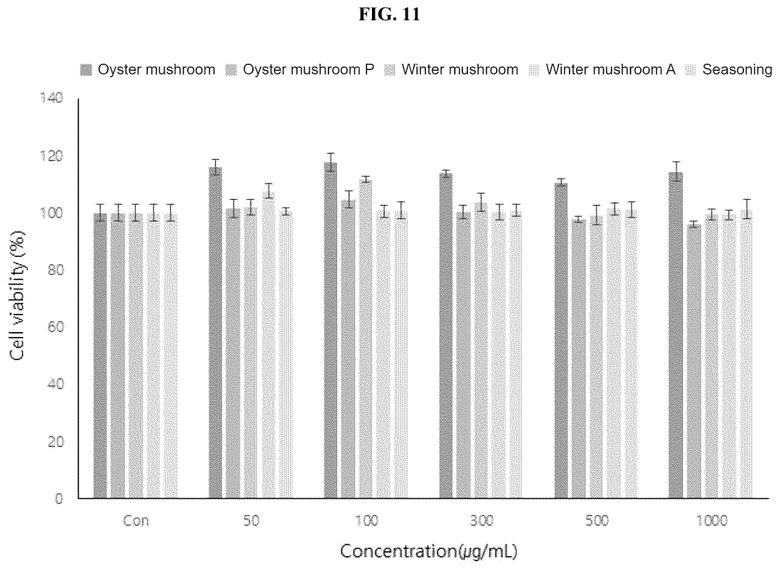

RBL-2H3 cells as a mast cell contain granules that are full of an inflammation mediator like histamine. When mast cells are activated and the inflammation mediator present inside the granules is released by degranulation, an inflammation response or an allergic response is caused. Result of analyzing cytotoxicity of the oyster mushroom extract, fermented product of oyster mushroom, winter mushroom extract, fermented product of winter mushroom, and natural seasoning on RBL-2H3 cells is the same as those illustrated in FIG. 11. Accordingly, it was found that no cytotoxicity on RBL-2H3 cells is exhibited by any test sample.

### 3) Result of Cytotoxicity Analysis for AGS Cells

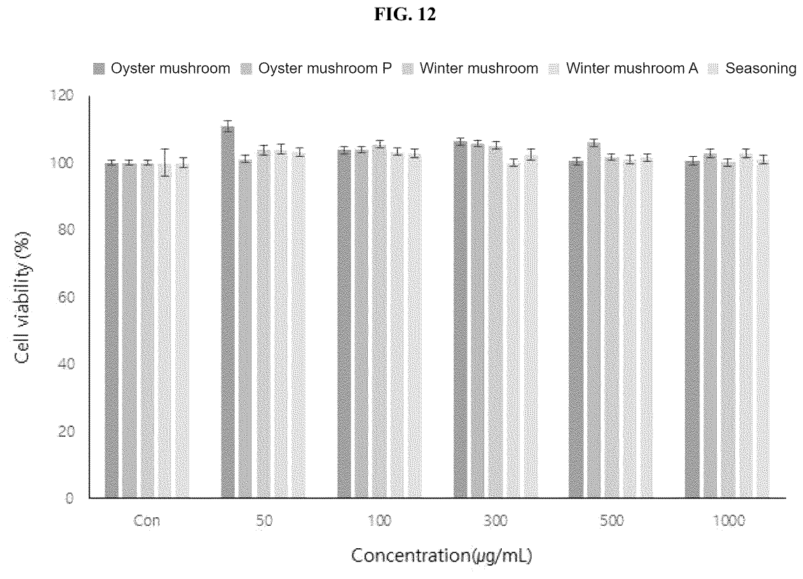

Result of analyzing cytotoxicity of the oyster mushroom extract, fermented product of oyster mushroom, winter mushroom extract, fermented product of winter mushroom, and natural seasoning on AGS cells is the same as those illustrated in FIG. 12. Accordingly, it was found that no cytotoxicity on AGS cells is exhibited by any test sample at any concentration.

### Example 13. Result of Analyzing Anti-Inflammatory Effect

### 1) Result of Analyzing NO (Nitric Oxide)-Inhibiting Effect

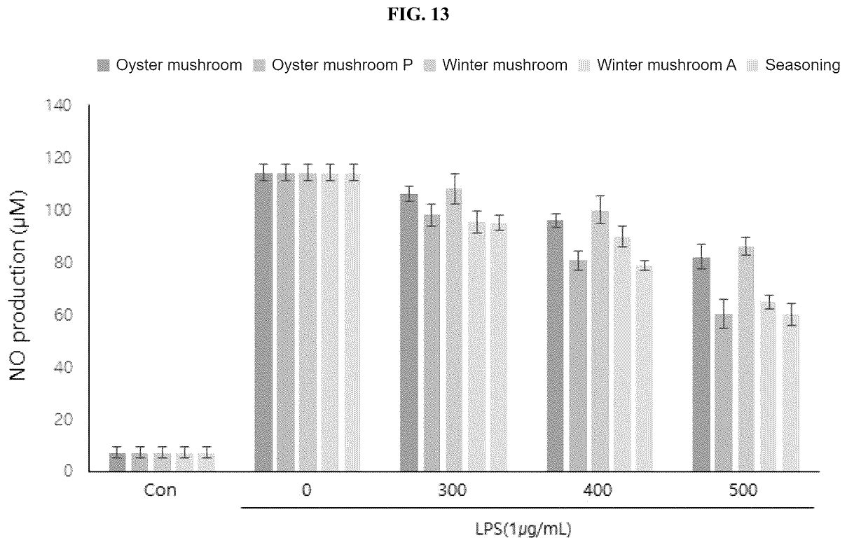

Result of analyzing NO (nitric oxide)-inhibiting effect of the oyster mushroom extract, fermented product of oyster mushroom, winter mushroom extract, fermented product of winter mushroom, and natural seasoning is illustrated in FIG. 13. At concentration of 500 μg/mL, the oyster mushroom exhibited the NO production amount of 82.0 uM, fermented product of oyster mushroom exhibited the NO production rate of 60.5 uM, winter mushroom exhibited the NO production rate of 86.0 uM, fermented product of winter mushroom exhibited the NO production rate of 64.9 uM, and natural seasoning exhibited the NO production rate of 60.1 uM. It was found from all test samples at each concentration that, compared to the non-processed ingredient, the production of NO (nitric oxide) is inhibited by the fermented product.

### 2) Result of Analyzing IL-1β-Inhibiting Effect

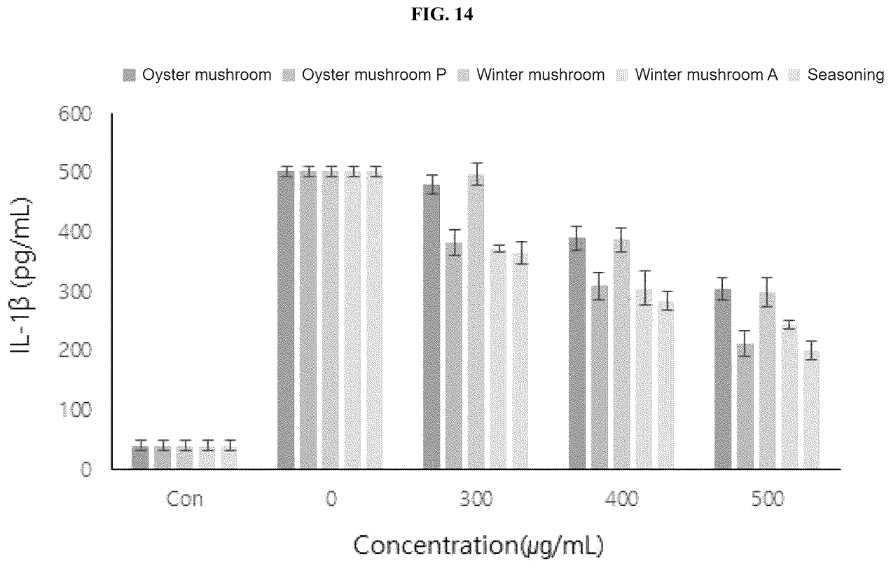

To examine the influence exhibited by cytokine on gene expression, IL-1β analysis was carried out. The main role played by IL-1 (Interleykin-1) is a mediator of immune response which is shown by a host in response to inflammations or other stimulations. It plays a key role in T cells, B cells, macrophage, or the like, and it is mainly involved in an immune response caused by an inflammation. Result of analyzing IL-1β-inhibiting effect of the oyster mushroom extract, fermented product of oyster mushroom, winter mushroom extract, fermented product of winter mushroom, and natural seasoning is illustrated in FIG. 14. At concentration of 500 μg/mL, the oyster mushroom exhibited the IL-1β production of 305.0 μg/mL, fermented product of oyster mushroom exhibited the IL-1β production of 211.0 μg/mL, winter mushroom exhibited the IL-1β production of 298.7 μg/mL, fermented product of winter mushroom exhibited the IL-1β production of 245.0 μg/mL, and natural seasoning exhibited the IL-1 production of 201.9 μg/mL.

### 3) Result of Analyzing TNF-α-Inhibiting Effect

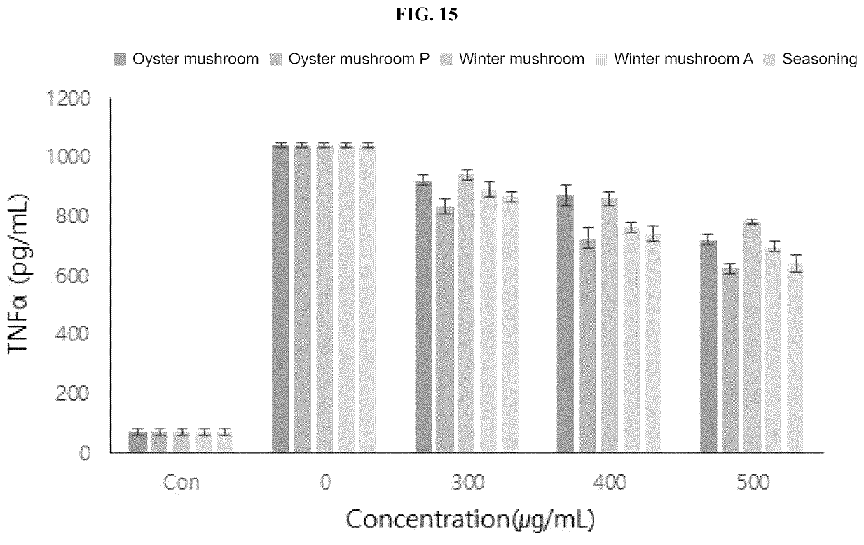

Result of analyzing TNF-α-inhibiting effect of the oyster mushroom extract, fermented product of oyster mushroom, winter mushroom extract, fermented product of winter mushroom, and natural seasoning is illustrated in FIG. 15. At concentration of 500 μg/mL, the oyster mushroom exhibited the TNF-α production of 720.0 μg/mL, fermented product of oyster mushroom exhibited the TNF-α production of 624.0 μg/mL, winter mushroom exhibited the TNF-α production of 781.0 μg/mL, fermented product of winter mushroom exhibited the TNF-α production of 698.0 μg/mL, and natural seasoning exhibited the TNF-α production of 641.0 μg/mL.

### 4) Result of Analyzing PGE2-Inhibiting Effect

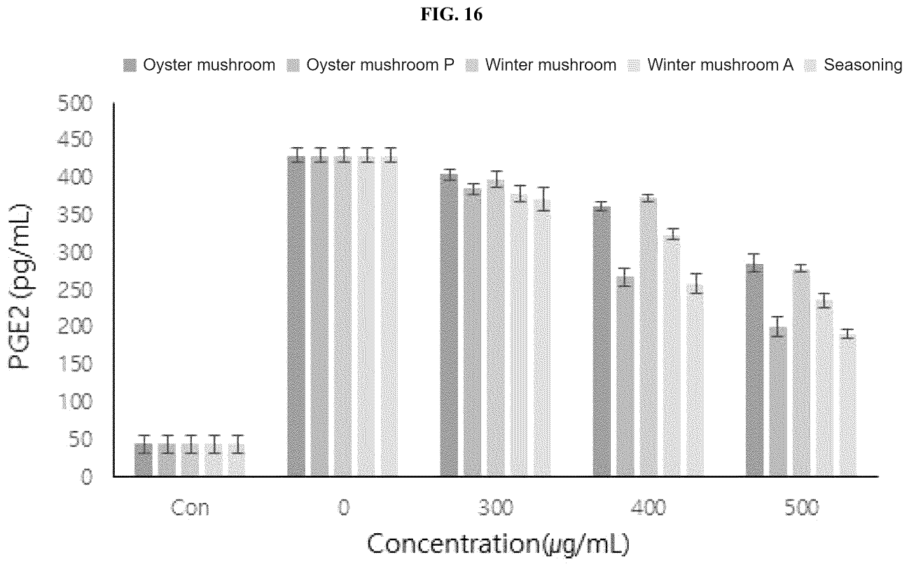

Result of analyzing PGE2-inhibiting effect of the oyster mushroom extract, fermented product of oyster mushroom, winter mushroom extract, fermented product of winter mushroom, and natural seasoning is illustrated in FIG. 16. At concentration of 500 μg/mL, the oyster mushroom exhibited the PGE2 production of 286.0 μg/mL, fermented product of oyster mushroom exhibited the PGE2 production of 201.0 μg/mL, winter mushroom exhibited the PGE2 production of 278.5 μg/mL, fermented product of winter mushroom exhibited the PGE2 production of 236.0 μg/mL, and natural seasoning exhibited the PGE2 production of 192 μg/mL.

### 5) Result of Analyzing β-Hexosaminidase

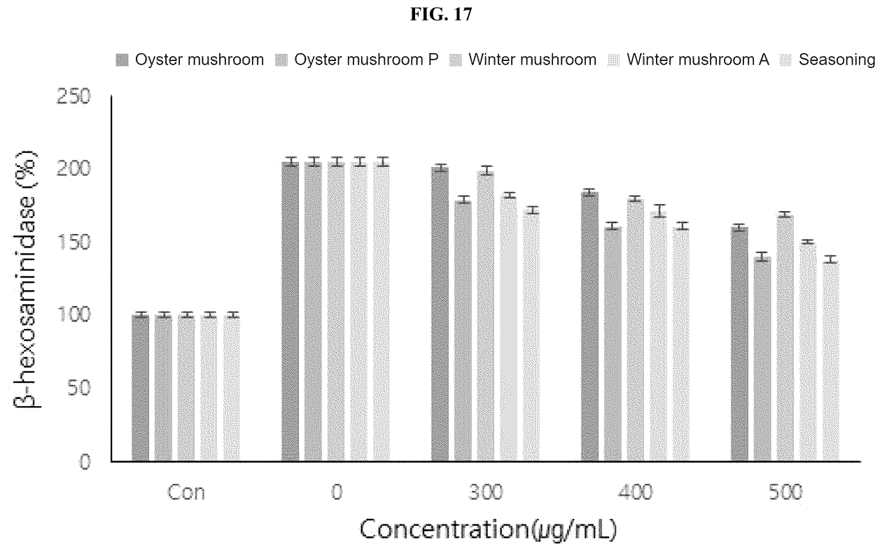

Result of analyzing β-hexosaminidase in the oyster mushroom extract, fermented product of oyster mushroom, winter mushroom extract, fermented product of winter mushroom, and natural seasoning is illustrated in FIG. 17. At concentration of 500 g/mL, the oyster mushroom exhibited the secretion ratio of 160.0%, fermented product of oyster mushroom exhibited the secretion ratio of 140.0%, winter mushroom exhibited the secretion ratio of 169.0%, fermented product of winter mushroom exhibited the secretion ratio of 150.0%, and natural seasoning exhibited the secretion ratio of 138.0%.

### 6) Result of Analyzing Histamine-Inhibiting Effect

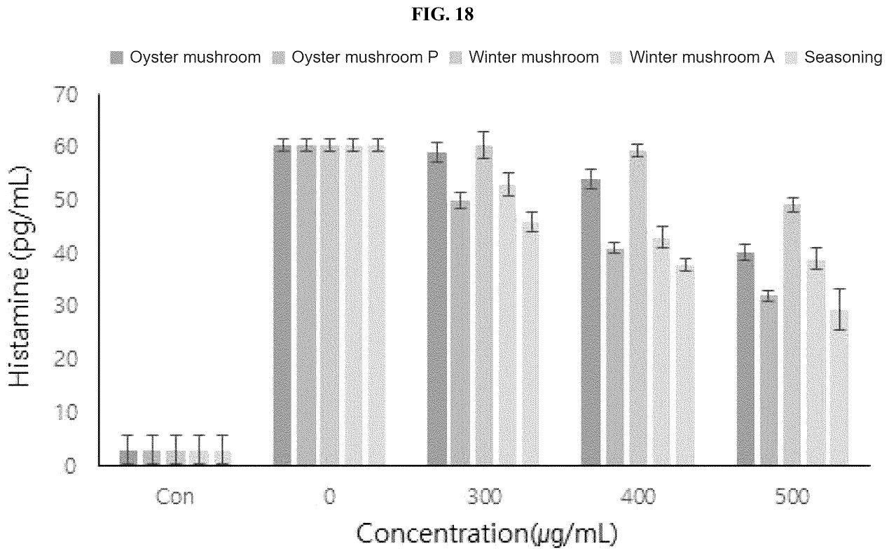

To determine the inhibitory effect on degranulation as an indicator of immediate allergic response, histamine secretion was measured. Result of analyzing histamine-inhibiting effect of the oyster mushroom extract, fermented product of oyster mushroom, winter mushroom extract, fermented product of winter mushroom, and natural seasoning is illustrated in FIG. 18. At concentration of 500 μg/mL, the oyster mushroom exhibited the histamine production of 40.3 pg/mL, fermented product of oyster mushroom exhibited the histamine production of 32.0 pg/mL, winter mushroom exhibited the histamine production of 49.2 pg/mL, fermented product of winter mushroom exhibited the histamine production of 39.0 pg/mL, and natural seasoning exhibited the histamine production of 29.5 pg/mL.

1: A method of producing natural seasoning, the method comprising:
(1) carrying out hot water extraction of kelp, followed by drying to prepare kelp extract;
(2) carrying out hot water extraction of oyster mushroom to prepare oyster mushroom extract;
(3) carrying out hot water extraction of winter mushroom to prepare winter mushroom extract;
(4) inoculating the oyster mushroom extract prepared in the step (2) with Pediococcus pentosaceus strain, followed by fermentation to prepare lactic acid bacteria fermented product of the oyster mushroom;
(5) inoculating the winter mushroom extract prepared in the step (3) with Lactobacillus acidophilus strain with Accession number of KCTC 14825BP, followed by fermentation to prepare lactic acid bacteria fermented product of the winter mushroom; and
(6) mixing the lactic acid bacteria fermented product of the oyster mushroom prepared in the step (4), the lactic acid bacteria fermented product of the winter mushroom prepared in the step (5), and the kelp extract prepared in the step (1).

2: The method of claim 1, wherein the Pediococcus pentosaceus strain is Pediococcus pentosaceus JMIL002 strain with Accession number of KCTC 14826BP and the Lactobacillus acidophilus strain is Lactobacillus acidophilus JMIL001 strain with Accession number of KCTC 14825BP.

3: The method of claim 2, wherein, in the step (1), the hot water extraction of the kelp is carried out for 2 to 4 hours at 75 to 85° C.;
in the step (2), the hot water extraction of the oyster mushroom is carried out for 3 to 5 hours at 75 to 85° C.;
in the step (3), the hot water extraction of the winter mushroom is carried out for 3 to 5 hours at 85 to 95° C.;
in the step (4), the Pediococcus pentosaceus strain is Pediococcus pentosaceus JMIL002 strain with Accession number of KCTC 14826BP;
in the step (5), the Lactobacillus acidophilus strain is Lactobacillus acidophilus JMIL001 strain with Accession number of KCTC 14825BP; and
the step (6) comprises mixing, based on the total weight of natural seasoning, 45 to 55% by weight of the lactic acid bacteria fermented product of the oyster mushroom prepared in the step (4), 27 to 33% by weight of the lactic acid bacteria fermented product of the winter mushroom prepared in the step (5), and 18 to 22% by weight of the kelp extract prepared in the step (1).

4: The method of claim 3, wherein, in the step (1), the hot water extraction of the kelp is carried out with 18 to 22 times (v/w) of water based on the weight of the kelp;
in the step (2), the hot water extraction of the oyster mushroom is carried out with 8 to 12 times (v/w) of water based on the weight of the oyster mushroom;
in the step (3), the hot water extraction of the winter mushroom is carried out with 8 to 12 times (v/w) of water based on the weight of the winter mushroom;
in the step (4), the fermentation is performed for 2 to 4 days at 34 to 40° C.; and
in the step (5), the fermentation is performed for 2 to 4 days at 34 to 40° C.

5: Natural seasoning produced by the method of claim 1.
